(19) United States
(12) Patent Application Publication
(10) Pub. No.: US 2009/0099485 A1
(43) Pub. Date: Apr. 16, 2009

(54) ULTRASOUND STANDING WAVE METHOD AND APPARATUS FOR TISSUE TREATMENT

(76) Inventors: Armen P. Sarvazyan, Lambertville, NJ (US); Boris Lagutin, Rostov on Don (RU)

Correspondence Address: BORIS LESCHINSKY, P.O. BOX 72, WALDWICK, NJ, 07463, US

(21) Appl. No.: 11/872,919

(22) Filed: Oct. 16, 2007

(51) Int. Cl. A61N 7/00

(52) U.S. Cl. 601/2

(57) ABSTRACT

Described herein are devices and methods for treatment of tissue with ultrasound standing waves. Vacuum-based or mechanical clamping resonators are proposed aimed at retaining tissue therewithin and in acoustic contact with ultrasound transducer means such as a single tubular piezotransducer or a pair of plane-parallel transducers. Ultrasound standing wave field is then applied at single or alternating resonance frequencies creating nodal patterns allowing expanding the area of treatment as compared with conventional devices. Real-time feedback is provided to monitor the progression of treatment. Additional device provisions include acoustic gel injector means, vacuum release means, indicator means for treatment completion, etc. This invention is particularly useful for non-invasive skin and adipose tissue treatments.

### FIELD OF THE INVENTION

The present invention relates generally to apparatuses and methods for noninvasive tissue treatments, and in particular to using ultrasound standing waves to cause local energy delivery to a target tissue area.

### BACKGROUND OF THE INVENTION

The noninvasive use of ultrasound for therapeutic or surgical treatment of internal tissues of a patient has been proposed in the art. A tissue can be exposed to ultrasonic energy in a focused or non-focused manner. When a non-focused transducer is used, all tissues located between the transducer and up to certain fading distance where energy levels are lower than the bioeffects threshold, are affected by the ultrasonic energy. When focused ultrasound is used, as a result of energy concentration, mainly the tissue at the focal range of the transducer is affected, while all other tissues between the transducer and the focus point or beyond are at least partially spared.

Systems and methods for performing a surgical, therapeutic or aesthetic medical procedure in target tissues of patient's body by using high intensity focused ultrasound (HIFU) are well known in the art. The HIFU systems are used for body aesthetic therapy by adipose tissue lysis as disclosed for example in U.S. Pat. Nos. 6,607,498; 6,645,162; 6,626,854; 6,071,239; all of which are incorporated herein by reference. Other similar terms used in the art include liposuction, lipoplasty and lipectomy. The main disadvantage of HIFU application for treatment of large volumes of tissue is small treated volume in lateral direction. For example, in the treatment of adipose tissue, which covers all body parts at an average thickness of 1-5 cm, the HIFU transducers are applied externally to the patient in the direction perpendicular to the body. To perform the treatment, the transducer needs to be moved step by step over many locations along the body and the procedure is greatly time consuming.

Various attempts to increase the size of treated area in HIFU systems were made. U.S. Pat. No. 6,071,239 discloses one example how the treated area is increased by applying HIFU simultaneously in multiplicity of discrete focal zones produced by a single transducer array. Other attempts to increase the size of the focal zone and thereby enlarge the treated area are described in U.S. Pat. Nos. 4,865,042; 6,613,004; and 6,419,648.

However, all of these techniques still appear to be effective only for treating a limited area of tissue as defined by a small size of a focal zone and are unsatisfactory for practical treatment of big areas of subcutaneous adipose or cellulite tissue regions without damaging other tissues.

Other disadvantage of conventional HIFU treatments of tissue is a restricted number of body areas suitable for treatment. Using of HIFU for adipose tissue treatment is restricted practically to include only an abdomen region, because of low fat thickness in other sites, complex body shapes, and close proximity of bones or vital organs elsewhere in the body.

Non-focused ultrasound systems are frequently used for therapeutic treatment of tissue at low ultrasound energy levels. However, an increase in ultrasound intensity for non-focused treatment of internal target tissues will lead to influence or damage of intermediate tissues, such as skin and superficial muscles. Non-focused ultrasound methods have been proposed for removing adipose tissue. One example of using non-focused ultrasound waves for disruption of the adipose tissue is disclosed in U.S. Pat. No. 5,884,631, issued to Silberg, however the technique according to this invention requires additionally injecting a special solution into the tissue prior to ultrasonic treatment.

Another example of using non-focused ultrasound waves for treatment of tissue is disclosed in U.S. Pat. Nos. 5,664,570 and 5,725,482 issued to Bishop, both of which are incorporated herein by reference in their entirety. According to these inventions, a plurality of standing ultrasonic waves is established in the tissue and the target tissue treatment volume is located at the common intersection of the axes of the standing waves. The drawback of this method is that there are very few areas on the body where the target tissue can be accessed simultaneously from all sides circumferentially, which is necessary for realizing this method. Although the method is based on non-focused ultrasonic waves, the treated volume is still small because it is limited to an area of intersection of a plurality of ultrasonic beams.

Another known example of tissue treatment using ultrasonic standing waves is facilitating wound healing as disclosed in the U.S. Pat. No. 6,960,173 issued to Babaev. Standing waves are used for creating ultrasonic radiation pressure, which increases the blood flow to wound area, stimulating healthy tissue cells and treating wounds.

The use of standing ultrasonic waves in combination with the HIFU treatment is disclosed in the U.S. Pat. No. 5,676,692 issued to Sangvi et al., though such a combination does not eliminate the drawback of focused ultrasound tissue treatment of a greatly limited volume of affected tissue.

The widest known field of biomedical application of ultrasonic standing waves is to manipulate biological cells in a solution or to separate different types of particles from a liquid or from each other. The use of a constant nodal pattern of a single ultrasound standing wave for particle capture and manipulation is described in detail for various patents listed below (these patents are all incorporated herein in their entirety by reference):

| | | | | |
|---|---|---|---|---|
| 4,055,491, | 4,280,823 | 4,398,925 | 4,523,632 | 4,523,682 |
| 4,673,512 | 4,759,775 | 4,877,516 | 4,879,011 | 5,006,266 |
| 5,527,460 | 5,613,456 | 5,626,767 | 5,688,406 | |

as well as in the U.S. Patent Application No. 2006037915 and international application No. PCT/AT89/00098.

Ultrasonic treatment of tissue aimed at body aesthetic therapy includes subcutaneous adipose tissue lysis as well wrinkle reduction and skin rejuvenation. The ultrasound energy focused in the dermis layer triggers a biological response that causes synthesis of new connective tissue in the dermis through activation of fibroblast cells. In U.S. Pat. No. 6,645,162, issued to Friedman et al., ultrasonic treatment of skin further includes detection of cavitation occurring in the focal zone, which is correlated to the extent of cell destruction.

The use of various useful feedback systems for controlling the dose of ultrasound energy applied to a patient's skin is disclosed in U.S. Pat. Nos. 6,113,559 and 6,325,769 issued to Klopotek. These feedback systems include temperature measurements on the surface of the skin, measurements of electrical conductivity of the skin, and detection of cavitation if the latter is the main mechanism of providing dermal irritation. In case of skin treatment, similar to that of subcutaneous adipose tissue for body aesthetic therapy, the known ultrasonic methods are time-consuming and not very efficient.

Therefore the need exists for new methods and devices aimed at treatment of large volumes of tissue, as for example in the case of removing significant amounts of adipose tissue from arbitrary body parts.

The need also exists for devices and methods for treating the skin and subcutaneous adipose tissue region using ultrasound energy, wherein the ultrasound energy is applied in a more efficient and safe manner.

### SUMMARY OF THE INVENTION

It is an object of present invention to provide improved devices and methods for noninvasive or minimally-invasive lypolitic, therapeutic or cosmetic treatment of large volumes of tissues including subcutaneous adipose or skin tissue on any desired body areas of patient using ultrasound standing waves.

For this purpose, the invention uses an ultrasonic resonator arranged to generate an ultrasound standing wave field at a single or multiple resonance frequencies in the target tissue temporarily positioned within that resonator for the duration of the treatment.

Useful treatment examples according to the invention include, but are not limited to: lysis of adipose tissue or cellulite, lipoma removal, skin rejuvenation, such as wrinkle and scar removal.

In one embodiment of the invention, the ultrasonic resonator is designed for vacuum suction of the target tissue to draw it inside the resonator, couple with an optional step of injecting of acoustic coupling gel into the tissue contact area.

In another embodiment of the invention, the ultrasonic resonator is designed for clamping of target tissue between plane-parallel surfaces containing one or two transducers.

In yet another embodiment of the invention, the resonator comprises a pair of equal-sized plane-parallel ultrasonic transducers.

In a further embodiment of the invention, the resonator is made in the shape of a suction cup and comprises a tubular ultrasonic transducer generating cylindrical standing waves in the tissue portion retained inside the resonator by temporary suction or using adhesive means.

In yet further embodiments of the invention, ultrasonic transducers are enhanced by providing quarter-wavelength-thick acoustic matching layers bonded onto the front surfaces of transducer elements and made from a material (such as some polymers) having an acoustic impedance matching that of soft tissue. This design allows for highly efficient transmission of acoustic energy into the target tissue. Most importantly, this layer also protects the skin contacting the transducers from damage, because the presence of such layer displaces skin from the pressure maximum points otherwise located at the boundary of the tissue.

The transducers of the ultrasonic resonator are activated by the control system, which drives them at frequencies in a range between a predefined minimum and maximum frequencies. These minimum and maximum frequencies are selected to include therebetween at least one resonance frequency (also referred to as a harmonic) of the target tissue retained within the resonator. A standing wave is formed in the tissue at each resonance frequency defining a particular nodal pattern associated with that particular frequency. Each resonance frequency defines a different nodal pattern at different locations throughout the tissue consisting of a plurality of pressure nodes and antinodes separated by an acoustic half-wavelength distance.

The tissue located in the ultrasonic standing wave field is affected by it with either one or both of thermal or non-thermal mechanisms, non-thermal mechanism including cavitational and various mechanical effects. Both mechanisms are most effective in the region of ultrasound pressure antinodes, which is the region of the pressure amplitude maxima. At these points distributed throughout the tissue according to the particular nodal pattern, two effects are most pronounced. First, at the minimum (most negative) acoustic pressure, the probability of forming cavitational microbubbles is the highest. Secondly, the generation of heat is maximal at the acoustic pressure amplitude maxima (K. Naugolnykh and L. Ostrovsky, Nonlinear Wave Processes in Acoustics. 308 pp., Cambridge University Press, 1998).

Switching the resonance frequency causes the nodal patterns of standing waves to change its locations. Therefore formation of ultrasound antinodes is encountered by different regions of tissue inside the resonator. Switching of frequencies therefore provides for even more uniform treatment coverage of the target tissue volume. The rate of frequency change is selected to be such that the duration of existence of each nodal pattern is sufficiently long to achieve necessary biological treatment effect, typically in the range of several seconds.

Further advantageous embodiments of the invention include an electronic control system, which automatically maintains over time the resonance oscillation in the resonator by continuously measuring the amplitude and/or phase of the signal at the driving piezotransducer. The measured data is used as a feedback signal to adjust the frequency when the resonance frequency of the resonator containing treated tissue is changed because of changes of acoustic properties of treated tissue. Acoustic properties of tissue may change either due to ultrasonically-induced structural damage or simply due to temperature change as a result of ultrasonic heating.

An essential element of the device of the invention is the ultrasonic transducer, which should preferably be selected to be a broadband transducer so that its driving at frequencies other than its own resonance frequency provides enough energy output into the resonator. The working resonance frequencies of the resonator should be selected to be preferably not too far away from the natural resonance frequency of the transducer as doing so may impede on the power output capability of that transducer. More sophisticated designs of the apparatus of the invention including variations of the control and feedback system and resonator design itself are described below in greater detail.

The preferred frequency range employed for treatment of tissues using standing ultrasonic waves is from about 0.1 to about 10 MHz, and the most preferred range is from about 0.2 to about 3 MHz. This range is defined first by the fact that the characteristic dimension of the tissue which needs to be treated is typically in the range from about a few millimeters to 4-5 cm. At the same time, the dimensions of the resonator should be selected from about half the wavelength of ultrasound to about tens of wavelengths of ultrasound to obtain a standing wave condition in the tissue placed within the resonator. In this range of frequency, the wavelength of ultrasound in aqueous solutions will be from about 15 mm down to about 0.15 mm.

In other embodiments of the invention, the resonator is formed by two plane-parallel piezotransducers. The electronic control system of the device is adapted to cause one transducer to be activated at the resonance frequency of the resonator while the second transducer is activated at a frequency oscillating about the frequency of the first transducer. Such frequency oscillation causes the nodal pattern to fluctuate its locations inside the resonator allowing treating an extended tissue region all at the same time. In a preferred embodiments, the range of frequency oscillation is about the halh-power bandwidth of the resonance peak.

In yet another embodiment of the device, the transducer and the electronic control system of the device form a phase-locked loop so that switching the driving signal frequency from one resonance frequency to another is simply achieved by inverting the phase of the signal at the output of the electronic system.

In further embodiments of the invention, the electronic control system provides real-time measurements of the changes of acoustical propagation parameters of tissues resulting from ultrasonic exposure and utilizes the obtained data for automatic optimization of the ultrasonic exposure parameters. Examples of such acoustic propagation parameters of tissue affected by the treatment are ultrasound velocity and attenuation in the tissue, which are assessed by measuring changes in the resonance frequency and the quality factor of the resonator containing the target tissue. Quality factor is a parameter characterizing the losses in the resonator and is defined as a ratio of the resonance frequency divided by the half-power bandwidth of the resonance peak. When ultrasound propagation parameters are reaching a predetermined threshold value, this indicates the completion of the treatment for each treatment zone of tissue. Operator indicator means may be then activated to prompt moving the device to another treatment zone of the tissue to continue treatment.

Other objects and features of the present invention will become apparent from consideration of the following description taken in conjunction with the accompanying drawings.

### BRIEF DESCRIPTION OF THE DRAWINGS

Non-limiting examples of embodiments of the invention are described below with reference to figures attached hereto that are listed following this paragraph. Identical structures, elements or parts that appear in more than one figure are generally labeled with a same numeral in all the figures in which they appear. Dimensions of components and features shown in the figures are chosen for convenience and clarity of presentation and are not necessarily shown to scale.

FIGS. 1A and 1B schematically show a prospective cross-section view of a first embodiment of an ultrasonic resonator made in the shape of a suction cup for vacuum-clamping a target tissue and producing cylindrical ultrasound standing wave field in the tissue;

FIG. 2A schematically shows a prospective cross-section view of second embodiment of a resonator made with a dual element ultrasonic transducer designed for mechanical clamping of a target tissue and producing an ultrasound standing wave field in the tissue;

FIG. 2B schematically shows a prospective cross-section view of one particular useful variation of the device shown on FIG. 2A with provisions for holding the device in a human hand;

FIGS. 3A and 3B represent block-diagrams of the entire system including a control system according to the third embodiment of the invention;

FIG. 4 is a block-diagram of the system according to the fourth embodiment of the invention;

FIG. 5 is a block-diagram of the system according to the fifth embodiment of the invention;

FIG. 6 shows frequency dependences of amplitude and phase of the signal at the output of the resonator shown on FIG. 5; and finally

FIG. 7 shows amplitude/frequency dependence of ultrasonic resonator in the presence of standing waves in the tissue filling the resonator.

### DETAILED DESCRIPTION OF THE PREFERRED EMBODIMENTS

A detailed description of the present invention follows with reference to accompanying drawings in which like elements are indicated by like reference letters and numerals.

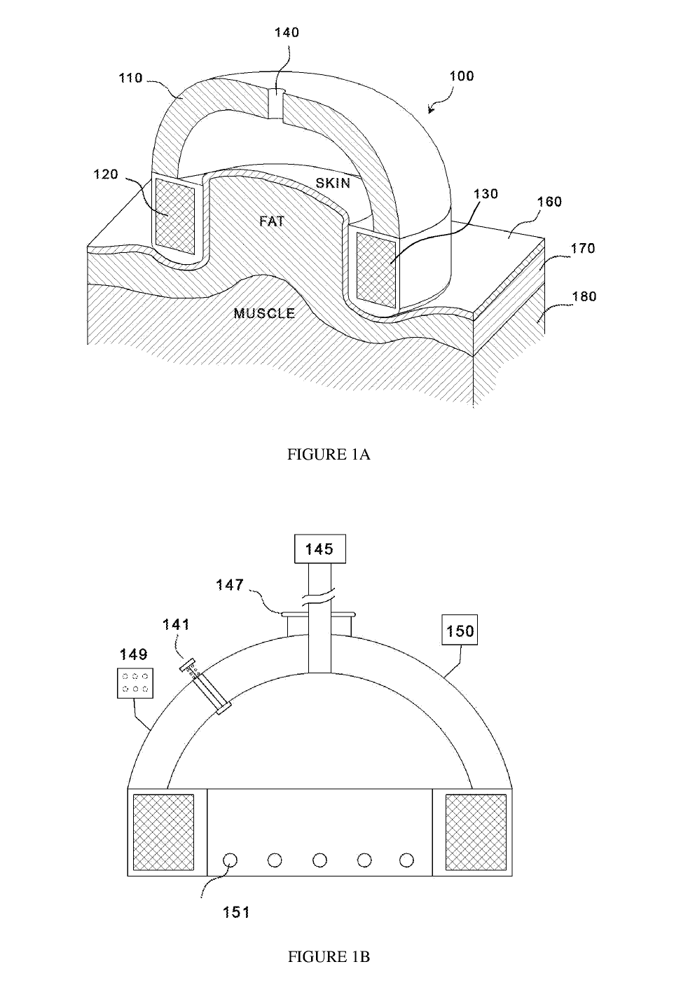

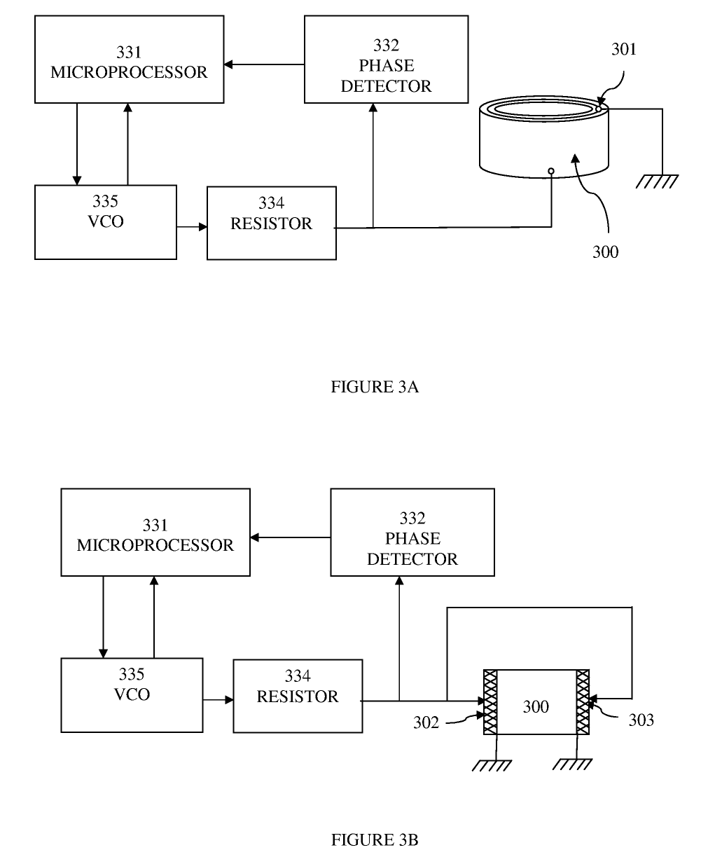

FIGS. 1A and 1B schematically show a prospective and side cross-section view of the first embodiment of the invention with a resonator 100 made in the shape of a suction cup. The resonator 100 is designed for vacuum clamping of a target tissue and for producing cylindrical ultrasound standing wave field in the tissue. The resonator comprises a tubular piezotransducer 120, which by way of example, may be made from PZT ceramics polarized in radial direction. The transducer may extend all the way about the periphery of the lower portion of the suction cup 110 and have an internal electrode and an external electrode as is shown later on FIG. 3A. Alternatively, as shown on FIG. 1A and schematically on FIG. 5, it can have a pair of opposite arch-shaped electrodes 120 and 130.

Vacuum-based tissue retaining means such as a suction cup 110 is equipped with means to apply vacuum through the opening 140, which may be located at the top of the cup but also may be located in other places. Known manual or automated vacuum supply means 145 (shown only schematically on FIG. 1B) may be employed to allow tissue to be drawn into the cup 110 and retained there for the time of treatment. Examples of manual vacuum supply means include syringes and squeeze bulbs. Automated vacuum source means may be designed to include electrical vacuum pumps. As with other known vacuum means designed for skin contact, the extent of vacuum may be limited to prevent injury to the patient.

Vacuum release means (shown schematically on FIG. 1B as a general position 141) may also be provided to allow tissue to be released from the cup 110. In its most simple configuration, a manual air vent button 141 may be used allowing vacuum to be relieved by introducing an outside air into the cup when the tissue needs to be released after treatment. The advantage of such vent button is that it is easy to operate by the user of the device. Such vent button can be ergonomically placed within easy reach of the hand of the user when holding the device during treatment as multiple uses of such button are envisioned during a single treatment session.

A more advanced configuration of the vacuum release means may involve a solenoid release valve activated by the control system when tissue release is needed. Further in this description, a feedback loop is described indicating to the operator that treatment of a particular tissue portion is complete. A vacuum release valve may be automatically activated to release the tissue from the resonator when the feedback loop has reached the threshold of treatment completion. The act of tissue release may by itself in that case be used as a signal of treatment completion aimed at indicating to the operator the need to move the device to another portion of the target tissue. It may also be a supplementary indicator when activated together with other direct indicators of treatment completion as described in more detail below.

The transducers may be covered by a suitable acoustic coupling gel (not shown) to achieve better transmission of acoustic energy into the tissue. Any kind of standard and medically approved acoustic contact fluid (generally referred to as gel) can be used (ultrasonic gel, water, oil etc). The quality of acoustic contact between transducer and tissue can be controlled by measuring of the transducer electric impedance Z (Z differs significantly with and without acoustic contact because of different level of mechanical loading of the transducer).

Such gel may be applied to the skin of a patient manually by the operator before or during the treatment. Alternatively, a gel injector means may be provided that are designed to inject the proper amount of coupling gel onto the tissue before or after drawing it into the suction cup. There may be one or more injector ports 151 located throughout the suction cup 110 and designed to ensure the proper distribution of the injected coupling gel over the skin of the patient. Injection means 150 are fluidly connected to the openings 151 and may be mechanical (squeeze button or syringe) or electrical (solenoid valve-activated motor-controlled or compressed gas-operated injection means) in which case activation of such injection means can be automated to be initiated when a fresh untreated portion of tissue is drawn into the cup and before activating ultrasound transducers. Another possibility for gel injection is that the same vacuum which is used to suck the tissue will be used to suck a gel from the lubricated skin or from a special container.

Depending on the size of the suction cup and the nature of the target tissue, the suction cup may retain various layers of tissue including skin 160, fat or adipose tissue 170 and muscle 180. The nature of the ultrasound signal applied by the transducers inside the resonator 100 is such that the therapeutic effect will only involve the target tissue as described in more detail below. In case of a lysis of adipose tissue for example, muscle tissue will not be not affected by the ultrasound while the adipose tissue is because only the latter will be easily sucked into the resonator.

The resonator 100 may also be equipped with an ergonomic handle 147 adapted for easy retaining of it in a human hand. It is important to provide easy retaining means allowing a good grip of the cup because during manipulation of the cup over the target tissue the user has to have full control of its position. The presence of the acoustic coupling gel may make it more difficult to retain the device in the hand of the user as it has a lubricating effect and may cause cup slippage. The handle 147 may serve as a convenient place to position controls 149 thereon such as a start/stop button, adjustment buttons, gel injection button, vacuum release button, indicator of treatment completion, visual or audio alarms, etc.

Also envisioned but not shown on the drawing is the connection cable extending from the resonator 100 towards a control system. This cable may contain electrical connection lines for ultrasound transducers and various controls including buttons and alarms. It may also contain vacuum pipe for tissue retaining means and pressure pipe for gel injector. In case the gel reservoir is located at the control system, the cable may also contain a gel pipe. Alternatively, the gel reservoir may also be incorporated with the suction cup itself.

To increase the acoustic output of the transducer, it could be useful to have acoustic matching layers between the transducer and the tissue. Ideally, the matching layer should be made a quarter-wavelength thick, but this condition can be satisfied to a limited extent because the frequency of the standing wave may vary in certain range.

When the matching layer is designed to have this thickness, it will also serve as a means for mitigating the risk of thermal damage of the skin in applications based on thermal mechanisms of tissue treatment. Having this layer to be quarter-wavelength thick will move the tissue away from the boundary of the transducer and therefore away from the area of pressure maxima locations. However, change of the frequency will result in the fact that wavelength of ultrasound in the matching layer material will also change and the thickness of the layer will became non-optimal. Further provisions to protect tissue from overheating include making the matching layer of a thermoconductive material, which can be thermostated or cooled by a Peltier element or by a flowing cooling fluid therethrough.

The specific design parameters such as resonator dimensions, clamping forces, ultrasound frequencies, details of mechanical clamping means, etc., strongly depend on specific clinical applications. The structure and design parameters of the system for big fat deposits treatment will obviously differ from those for cellulite or skin treatment.

There are numerous factors limiting the physical dimensions of the resonator 100:

Obviously, it is difficult to retain very small portions of tissues (less than a few mm thick) and it is also hard to clamp big portions of tissue (more than about 5-6 cm thick) because of anatomical limitations, elasticity properties of tissue and patient's discomfort and pain limit. That defines generally the range of sizes for the device;

To form a standing wave pattern, it is necessary to limit the spread of ultrasonic beam and limit the distance between transducers in the resonator 100 to not be large as compared with the width and height of the transducers;

Another critical issue regarding the resonator 100 size is related to the choice of the frequency range optimal for a particular mechanism of tissue treatment. To produce a cavitational effect, lower frequencies are employed, where the ultrasound wavelength is in the range of millimeters and more. To produce a thermal effect, higher frequencies are used where the wavelength is less than a few millimeters. At the same time, the ratio of the acoustical path to the ultrasound wavelength should not be too high (preferably less than 10) to efficiently form a standing wave.

The dimensions of the cup 110 and the resonance frequency of the transducers 120 and 130 should be different for different body parts, sizes and applications. Generally, other tissue retaining means have been designed using the guidance of not exceeding a force on the tissue to be greater than about 1-10 kg in case of the clamping surface area of about 2-10 cm³, depending on application and specific body part.

Preferred dimensions of vacuum tissue retaining means based on cylindrical resonator 100 as shown on FIG. 1 are as follows:
height 10-25 mm and the cylindrical resonator internal diameter of 30-60 mm when used preferably for abdomen big fat deposits treatment; and
height 3-10 mm and the cylindrical resonator internal diameter of 10-30 mm when used preferably for small fat deposits treatment; face cosmetics, cellulite and skin treatment.

In use, the device is initially brought in close proximity with the first treatment zone of the target tissue of the patient. The vacuum-based tissue retaining means are activated such that a portion of tissue located under the suction cup 110 is drawn into it. Coupling gel is optionally applied either before drawing of tissue into the cup 110 or inside the cup using means as described above. The ultrasound transducer means in then activated. The transducer 120 (or transducers 120 and 130) is driven by the control system using at least a single or optimally multiple-frequency method of sonication switching the driving frequency between several resonance frequencies of the tissue as will be described in more detail later. Importantly, the transducer is driven during a predefined period of time at at least one resonance frequency of tissue inside the resonator 100. Such resonance frequency causes appearance of a standing wave and therefore appearance of a particular nodal pattern with locations throughout the tissue defined by this particular resonance frequency. Antinodal acoustic pressure minima and maxima locations will define the plurality of places where cavitation and heating of tissue are the most pronounced causing the desired therapeutic effect in such locations. The driving frequency of the transducers is then optionally changed to another resonance frequency with a different nodal pattern, causing desired therapeutic effects to happen in a new plurality of locations. Changing of transducer frequency therefore causes treatment to occur more evenly throughout the portion of tissue retained inside the resonator 100. Once the treatment is complete, the user moves the resonator 100 to another treatment zone and repeats the treatment cycle again eventually covering all treatment zones of the target tissue.

Treatment time is typically about 1-5 sec at each treatment zone, depending on application and mode of ultrasound, which could be either CW (continuous wave) or pulsed. CW mode is preferable for the thermal treatment of the tissue, while cavitation-based treatment might be optimal with pulse mode. The pulse duration should be long enough to form a standing wave. Typically, at least 20-30 periods of ultrasonic oscillations are needed to generate an effective standing wave. Duty cycle in the range of 1/10 to about 1/100 could be sufficient to induce cavitation without significant heating of the tissue.

Treatment of tissue in each position of the treatment zone can be done in one or two and more steps. Ultrasonic pressure nodes locations where the tissue is affected by a thermal mechanism, cavitational mechanism, or by the combination of both mechanisms, cover only a fraction of the volume of tissue that needs to be treated. As mentioned above, by switching the harmonics of standing wave that is by changing the nodal pattern of standing waves, different regions of the tissue are treated. The duration of each step corresponding to a particular harmonic of standing wave frequency is selected to be such that the time of existence of each nodal pattern is sufficiently long to achieve necessary therapeutic effect, typically on the order of a second. In certain embodiments of the method of this invention, more than two steps may be used. After the second ultrasonic exposure, the control system again switches the frequency of the ultrasound transducer to yet another resonance frequency, such as the back to first resonance frequency or to a third resonance frequency.

A further important feature of the method of this invention is the ability to verify accomplishing the desired effect or monitoring treatment progression in real time. The electronic control system, which provides automatic adjustment of the standing wave condition, is adapted not to allow the system to be driven out of resonance. It also provides real-time assessment of the changes of ultrasound velocity and attenuation in tissue resulting from ultrasonic exposure. Continuous assessment of tissue acoustic propagation parameters is made for example by measuring changes in the resonance frequency and the so-called Q-factor (quality factor) of the resonator containing the tissue. Changes in resonance frequency linearly depend on the changes in the ultrasound velocity in tissue. Q-factor characterizes the attenuation of ultrasound in tissue and is defined as a ratio of the resonance frequency divided by the half-power bandwidth of the resonance peak. Q-factor is inversely proportional to the total energy loss in the resonator 100.

Evaluation of acoustic propagation parameters of tissues and liquids placed in ultrasonic resonator is generally disclosed in the U.S. Pat. No. 5,533,402 issued to Sarvazyan and Ponomarev and incorporated herein by reference. Ultrasound velocity and attenuation provide information on tissue structure and composition including water and protein content (Sarvazyan A P, Hill C R, Physical chemistry of the ultrasound-tissue interaction.-In: Physical Principles of Medical Ultrasonics, Chapter 7, eds. C. R. Hill, J. C. Bamber and G. R. ter Haar., John Wiley & Sons, 2004, 223-235; and Sarvazyan et al., Ultrasonic assessment of tissue hydration status.-Ultrasonics, 2005, 43(8), 661-71). Both the velocity and attenuation of ultrasound are frequently used to monitor processes in biological tissues and fluids (Sarvazyan A P, Ultrasonic velocimetry of biological compounds.-Annu. Rev. Biophys. Biophys. Chem., 1991, vol. 20, 321-342).

Most importantly, measurements of resonant frequency and the Q-factor of the resonator 100 allow not only assessment of the lesion formation being produced by ultrasound in tissue, but also monitoring of factors affecting the tissue, such as temperature increase and onset of cavitation. Ultrasound velocity in tissue is temperature-dependent, therefore heating of tissue, even before it is clinically affected by heat, results in the change of the ultrasound wavelength, and, consequently, in the change of the frequency of standing wave.

In the case when cavitation is the desired mechanism affecting the tissue, ultrasound absorption in the resonator 100 immediately increases as soon as cavitation bubbles appear in the tissue, even before a significant thermal damage of tissue is produced. Evaluating changes in the Q-factor of the resonator 100 allows quantitative monitoring of the cavitation onset in the tissue. These changes of the Q-factor can be detected by assessment of phase-frequency slope or the half-power bandwidth of the resonance peak.

Therefore heating of tissue, initiation of cavitation bubbles or combined effect of both mechanisms can be detected by evaluating the parameters of the resonance peak.

According to the present invention, the areas of tissue affected by high intensity ultrasound are more uniform and do not have such sharp gradients as in case of conventional focused ultrasound. Areas of tissue affected by ultrasound according to the invention coincide with the extended regions of acoustic pressure maxima defined primarily by the nodal pattern of the standing wave, making the device safer in use by preventing sharp peaks in temperature rise.

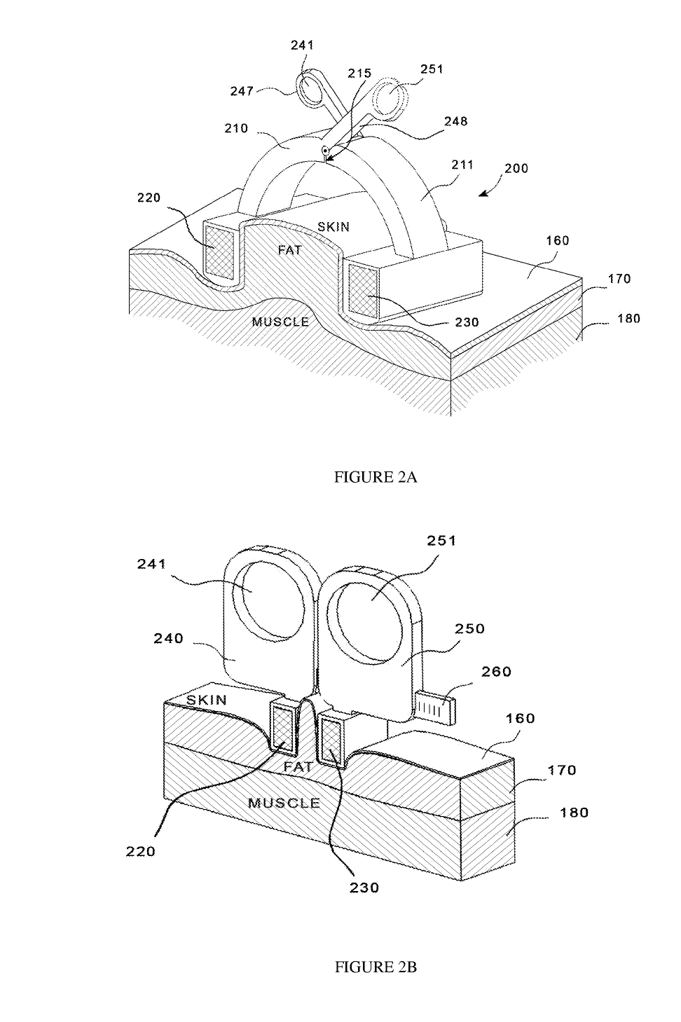

FIG. 2A schematically shows a prospective cross-section view of a second embodiment of the invention including a resonator 200 designed for mechanical retention of a target tissue between two transducers 220 and 230. The transducers may also be supplemented by quarter-wavelength-thick acoustic matching layers directly bonded onto the front surfaces of the transducers and made from a polymer material.

The resonator 200 includes a tissue retaining clamping means including a first arm 210 hingedly connected at its top end with a top end of a second arm 211. Plain-parallel transducers 220 and 230 are attached at respective bottoms of the arms 210 and 211. Swinging the arms 210 and 211 open allows the resonator 200 to be placed on target tissue while swinging the arms close will draw the tissue into the resonator volume and between the transducers 220 and 230. Vacuum suction may also be alternatively used to draw tissue between the plane-parallel transducers.

The mechanical details of clamping the tissue performed by means of the second embodiment of the invention are similar to other clamps known in the art such as a medical pincer, carpenter vice, jaw vice, clothes peg clamp etc. These or similar mechanisms can be deployed to control the proper movement of the arms of the clamp. When the arms are in their closed position, it is important to ensure that the facets of the transducers are parallel to each other, also meaning that the clamp has to have the same distance between the transducers each time it is closed. This can be achieved by providing for example a mechanical stop means 215 between the arms such that they are moved to the closed position until they hit that stop. At the same time, due to concerns about tissue damage caused by excessive forces (as described above), provisions are envisioned to prevent such excessive clamping. Such provisions include among others spring-biased support for the transducers, spring-biased limiters for arms closure, spring-biased indicators of excessive force causing extension of red tags for example when excessive force is applied etc.

One- or two-part handle 247 and 248 can be provided above or below the level of the hinge between the arms so as to make it convenient to grab the resonator 200 and retain it in one hand while manipulating it to close and open the arms 210 and 211. Finger openings 241 and 251 may be for example provided to ease the handling of the device.

Other supplemental means may also be included in the design of the second embodiment of the invention as described above for the first embodiment of the invention including gel injectors, alarm indicators, controls incorporated with the handle, etc.

The choice of resonator parameters for the second embodiment of the invention depends on the chosen transducer resonance frequency, and vice versa. The distance between facets of the plane-parallel transducers 220 and 230 could not be less than a half-wavelength of ultrasound in tissue and it also could not be more than about 10 half-wavelength of ultrasound in tissue. Since speed of sound c in all soft tissues does not vary much and is typically within 1550 m/s±150 m/s, there is a simple relationship between the frequency f and the wavelength of ultrasound in tissue measured in mm, which is roughly proportional to 1550/f(kHz).

Preferred dimensions of tissue retaining clamping means based on plane-parallel transducer resonator 200 and in view of the restrictions described above for the first embodiment of the invention are as follows depending on a particular application:
height 15-30 mm, length 30-100 mm, distance between facets 20-50 mm, as used preferably for abdomen big fat deposits treatment;
height 5-15 mm, length 10-50 mm, distance between facets 10-20 mm, as used preferably for small fat deposits treatment;
height 5-10 mm, length 10-30 mm, distance between facets 2-10 mm, as used preferably for cellulite and skin treatment.

FIG. 2B shows a useful variation of the second embodiment of the invention when sliding means for opening and closing of the resonator are employed. The first arm 240 has an opening 241 for placing at least one finger therethrough and retaining the device in a hand of the user. It also contains a slider 260 extending towards the second arm 250 with its corresponding opening 251. The arm 250 has an internal opening adapted to slide over the slider 260 making it possible to open and close the resonator 200 with one hand while retaining control over its position. As mentioned before, this version may also have a mechanical stop in place to prevent tissue pinching and excessive clamping. However, the significant advantage of this arrangement is that transducers are always retained in a plane-parallel relationship to each other. This design also allows for some variation of the distance between the facets of the transducers. It is compensated for by the control system in terms of still providing for transducers activation at resonance frequencies to ensure the presence of standing waves in the tissue clamped therebetween.

Another provision of this design is that the distance between the transducers when both arms are brought together in closed position is selected such that tissue damage is prevented according to the general size recommendations mentioned above.

Referring to FIGS. 3A and 3B, there are shown block-diagrams of the control system according to the third embodiment of the invention. FIG. 3A shows the control system for driving a tubular ultrasound transducer 300 having an inside grounded electrode 301, this system is described now in more detail.

Transducer excitation alternating current signal is preferably generated by a voltage controlled oscillator (VCO) 335. A microprocessor 331 is used to set the voltage, which is sent out to VCO 335 and defines the frequency of the alternating current electrical signal. The output of the VCO 335 is sent to the ultrasound transducer 300 via a complex resistor 334. The complex resistor 334 acts as a voltage divider and splits the electrical signal proportionally so that it could be utilized for detecting changes of the impedance of the transducer 300 acoustically loaded by the ultrasonic resonator.

The exact information about resonance frequencies of the tissue inside the resonator may not be available at the beginning of operation of the device since these frequencies are defined by the speed of sound in the tissue as discussed above. Therefore the control system is made capable to automatically detect these resonance frequencies by measuring changes of electrical impedance of the transducer 300. When a standing wave is established in the resonator containing target tissue, the acoustical loading of the transducer 300 changes, thus affecting its electrical impedance. Every time when the driving frequency of the transducer 300 is approaching the resonance frequency of the tissue-filled resonator, the amplitude and the phase of the signal at the output of the complex resistor 334 changes significantly. These changes are detected by the amplitude and/or phase detector 332 and sent back to the microprocessor 331 indicating the appearance of standing waves at certain detected resonance frequencies.

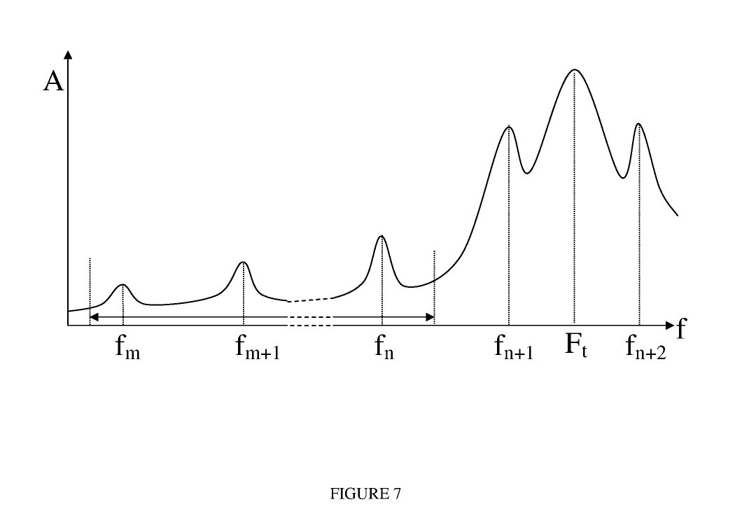

Although as stated above, exact resonance frequencies may not be known at the beginning of the operation of the device, their approximate values can be estimated knowing the general geometry of the resonator. It is useful to select the minimum and the maximum frequency of the initial sweep to cover at least one and preferably several harmonics of the resonator. At the same time, it may be best to not include the natural resonance frequency of the transducer 300 in this range, which may cause uneven levels of ultrasound intensity in the successive standing wave patterns in the multiple-frequency mode of sonication, as discussed in more detail below for FIG. 7.

A further improvement of the method of the invention includes repeating from time to time a sweep of frequencies to refresh the current values for the set of resonance frequencies as well as to determine if the new set has deviated from the previously recorded values of resonance frequencies. Detecting a change in the amplitude and/phase of the signal obtained by the detector 332 indicates the presence of changes in tissue positioned inside the resonator, such as a completion of lysis or tissue temperature increase, which affected the position of the resonance frequencies. Once the change reaches a predefined threshold, the operator is notified about the treatment completion and the device may be optionally turned off until the next treatment zone is available for treatment. One useful safety provision is to measure the tissue ultrasound propagation parameters before each treatment and compare it to the previously recorded value obtained for the previously treated zone of tissue. If the value is not different the treatment is not initiated to avoid treating the same tissue portion twice.

The above described frequency sweep may be conducted either over the entire frequency range covering all resonances used for treatment of tissue, or preferably only in the vicinity of the resonance frequencies obtained during the initial sweep. Since the microprocessor 331 is adapted to continuously monitor the resonance frequencies using the driving signal provided by detector 332, any shift of the resonance frequency is detected at an early stage. This means that only small corrections of the recorded values of the resonance frequencies are needed and there is no need to repeat a complete diagnostic sweep such as the one conducted at the beginning of the procedure. Making small local sweeps in the vicinity of the maxima of the previously recorded resonance peaks is sufficient to maintain effective operation of the device.

These repeated sweeps allow to accurately maintain the standing wave condition in the stepwise mode of sonication and do not affect the procedure time of tissue treatment because they take negligible time. The time for each such adjustment sweep is on the order of a millisecond while the typical times needed for the sonication procedure is on the order of seconds and minutes. These repeated sweeps provide automatic detection and control of the standing wave condition in the resonator independent of variations of temperature. The magnitude and/or timing of adjustments that need to be made to maintain the resonance conditions in the tissue filling the resonator can be used as a quantitative measure characterizing changes in the tissue, such as temperature increase or progression of lysis. Excessive heating of the tissue may therefore be avoided when increase in temperature is detected early enough by automatic adjustment of the ultrasound intensity.

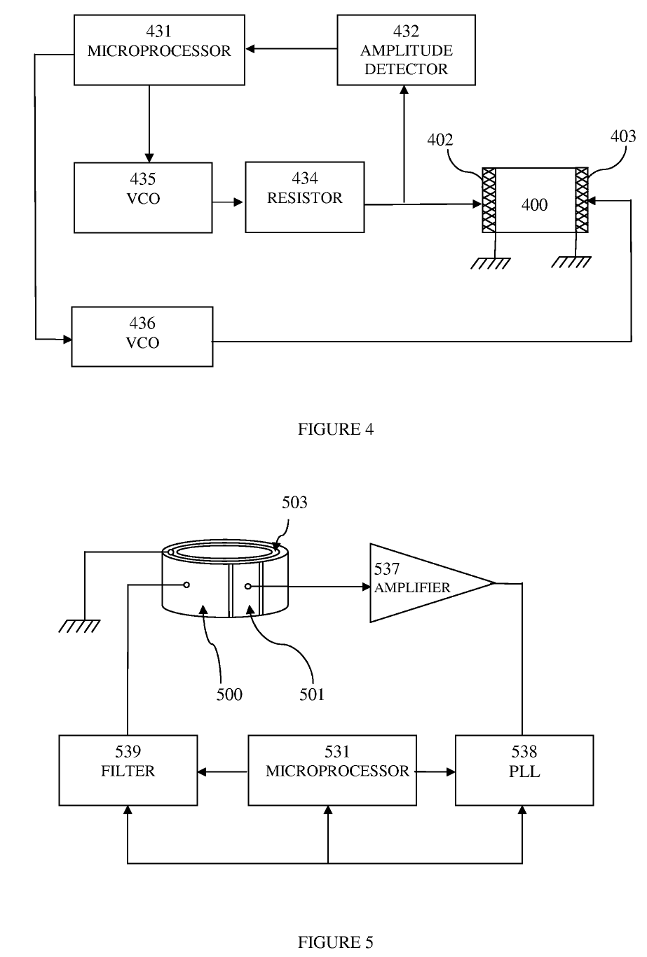

While the control system shown on FIG. 3A (or FIG. 5 as will be apparent from the later description) can be advantageously used with cylindrical resonators of the first embodiment of the invention, other resonators such as having plane-parallel transducers can be advantageously driven by other configurations of the control system as for example depicted on FIGS. 3B and 4.

FIG. 3B shows a variation of the system shown on FIG. 3A in which the transducer means 300 comprises a pair of plane-parallel transducers 302 and 303 and the driving signal is applied to both of these transducers simultaneously. The rest of the system works in a manner similar to that depicted in FIG. 3A.

FIG. 4 shows a fourth embodiment of the invention, which preferably uses a transducer means 400 comprising a pair of plane-parallel transducers 402 and 403 but each of these two transducers is driven individually by a dedicated voltage-controlled oscillator (VCO) 435 and 436. Each VCO is being controlled by the microprocessor 431. The frequency and phase of the signal generated by the VCO 436 driving the transducer 403 could be the same or preferably oscillating back and forth about the frequency generated by the VCO 435 and applied to the transducer 402. As described above, the feedback circuit consisting of the complex resistor 434 and phase and amplitude detector 4132 provides for automatic monitoring of required mode of the frequency generation of the signal applied to the transducer means 400. At the same time, the variation of the frequency or amplitude of the signal applied to the transducer 403 provides for a possibility to slightly shift or to oscillate in space the locations of nodal patterns of the standing wave. This shift of locations within the same nodal pattern may allow to increase the efficacy of tissue treatment when a stepwise sonication method is applied at a lower rate of switching between resonance frequencies.

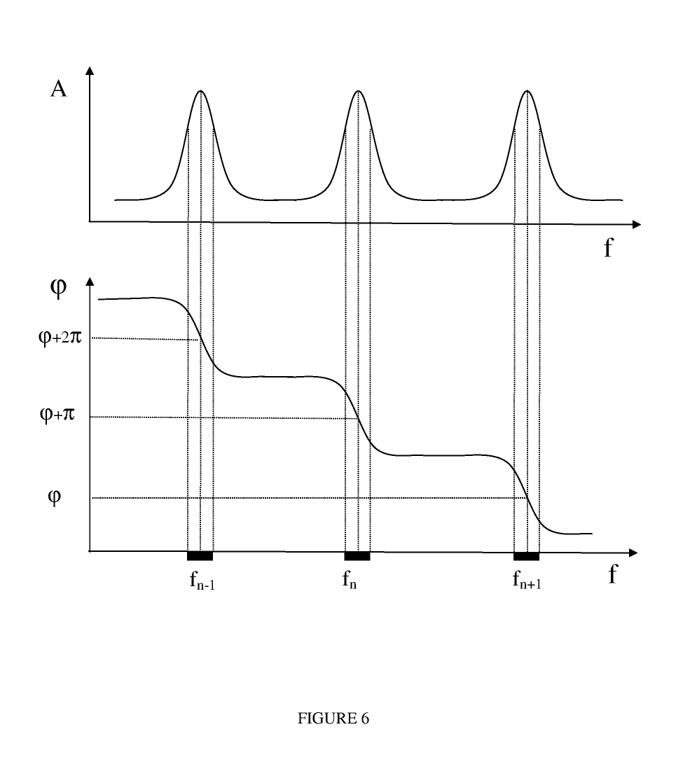

FIG. 5 shows a schematic block-diagram of the fifth embodiment of the invention. In the device according to this embodiment of the invention, an ultrasonic resonator 500 is formed by two piezotransducers 501 and 503 and is connected to a simple oscillation and feedback control system, including a broadband amplifier 537, a phase-locked loop chip 538, a microprocessor 531 and a bandpass filter 539. The transducer 501 serves both as a reflector and a receiver of ultrasound. FIG. 6 shows frequency dependences of amplitude and phase of the signal at the receiving transducer 501 in a frequency band covering several resonance harmonics fₙ₋₁, fₙ, and f_{n±1}. The phase of the signal from the receiving transducer 501 is changed by 180° when the frequency is swept through a region corresponding to a resonance peak marked by bold lines on the frequency axis of the graph of FIG. 6. As seen in FIG. 6, the inflection point of the phase/frequency curve corresponds to the maximum of the resonance peak that is optimum frequency for generating a standing wave in the resonator.

Maintaining phase relationships between transmitted and received signals close to the value corresponding to the inflection point of the phase characteristics provides necessary conditions for generation of standing wave. The phase-locked loop (PLL) chip 538 is adapted to automatically maintain the resonance phase relationship between the input and output signals of the resonator 500 by changing the oscillation frequency. The circuit maintains the appropriate phase relationship and therefore maintains resonance conditions despite variations in temperature or other conditions that alter the sound velocity in the treated tissue. The resonator 500 functions as the frequency-determining element of the oscillator. Constraining the oscillator to operate in the specific frequency region by adjusting the bandpass of the amplifier 537 allows one to generate a standing wave corresponding to the chosen harmonic of the resonator.

To switch the frequency, that is to move from one harmonic of the resonance to another, the microprocessor 531 is designed to vary the voltages controlling either the setting of the bandpass filter 539 or the setting of the phase of the PLL circuit 538.

FIG. 7 shows a typical amplitude/frequency dependence of an ultrasonic resonator in the presence of standing waves. The horizontal solid arrow denotes a frequency region, which includes several harmonics in the treated tissue placed in the resonator, from fₘ to fₙ, and which is appropriate for multiple-frequency mode of tissue sonication according the methods of the current invention. The working frequency range should preferably not include the exact resonance frequency of the transducer Fₜ because in that case the neighboring harmonics of standing wave in tissue may greatly differ in the amplitude and consequently in the level of energy delivered to tissue.

The above described main applications of the invention are for use with skin, such as cellulite and subcutaneous fat treatment. However, the present invention can also be used for other applications. One area of such applications includes incorporating the tissue retention means of the invention on a device adapted to be inserted through a natural body opening such as intravascularly, in colon, in rectum or vagina. Further downsizing a cup-shaped resonator allows incorporation thereof with various catheter-like devices. Tissue treatment using such version of the invention may include lysis or destruction of target soft tissues located in the vicinity of such natural openings and channels. These variations of the invention may find practical application for a number of procedures that are performed today with more invasive surgical means. Examples of clinical applications using these devices include among others such procedures as polyp removal in colonoscopy, endovaginal and tracheal therapy, etc.

Although the invention herein has been described with respect to particular embodiments, it is understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method for tissue treatment comprising a step (a) of applying a first ultrasound standing wave field to said tissue defining a first nodal pattern within said tissue; said method further including maximizing the effect of said treatment by adjusting an ultrasound wave frequency to maintain a condition of resonance and said first standing wave field, the magnitude of such adjustment is used for monitoring progression of treatment.

2. The method as in claim 1 further comprising the following steps:
(b) applying a second ultrasound standing wave field to said same tissue defining a second nodal pattern, said second nodal pattern having different nodal locations from that of said first nodal pattern, said method further including maximizing the effect of said treatment by adjusting the ultrasound wave frequency to maintain said condition of resonance and said second standing wave field.
(c) repeating steps (a) and (b) until completion of said treatment.

3. (canceled)

4. (canceled)

5. (canceled)

6. The method as in claim 1 wherein the step of adjusting said ultrasound wave frequency to maintain the condition of resonance is periodically repeated during the treatment of said same tissue.

7. The method as in claim 1 wherein said step of adjusting said ultrasound wave frequency includes comparing a current ultrasound frequency with previously defined resonance frequency and a shift in said resonance frequency is used as a real-time feedback signal characterizing the state of tissue and progression of treatment.

8. The method as in claim 1, wherein said first ultrasound standing wave field is applied at a frequency, which value is oscillating about said resonance frequency.

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. The method as in claim 1 wherein the step of adjusting said ultrasound wave frequency to maintain the condition of resonance is conducted continuously during the treatment of said same tissue.

27. The method as in claim 1 wherein said ultrasound standing wave is a cylindrical standing wave.

28. The method as in claim 1 wherein said tissue is accessed through a natural body opening.

29. The method as in claim 1 wherein said maintaining of said condition of resonance is achieved by using a phase-locked loop method.

30. The method as in claim 1 wherein the progression of treatment is assessed by evaluating a quality factor of a resonance peak.
